(19) United States
(12) Patent Application Publication
(10) Pub. No.: US 2024/0279584 A1
(43) Pub. Date: Aug. 22, 2024

(54) BIOREACTOR INTERFACE PLATE

(71) Applicant: Oribiotech Ltd, Greater London, London (GB)

(72) Inventors: Farlan Veraitch, Greater London, London (GB); William Raimes, Greater London, London (GB); Martin Hoole, Melbourn Hertfordshire (GB); Jason Palmer, Melbourn Hertfordshire (GB); Nicholas Harding, Melbourn Hertfordshire (GB); Hector Fraser, Melbourn Hertfordshire (GB)

(21) Appl. No.: 18/293,531

(22) PCT Filed: Jul. 27, 2022
(86) PCT No.: PCT/GB2022/051967

§ 371 Date: Jan. 30, 2024

(30) Foreign Application Priority Data
Jul. 30, 2021 (GB) 2111042.4

(51) Int. Cl. C12M 1/00; B01D 35/027; B01D 35/05; C12M 1/09

(52) U.S. Cl. CPC C12M 29/04; B01D 35/027; B01D 35/05; C12M 23/26; C12M 23/38; C12M 23/56

(57) ABSTRACT

A bioreactor interface plate assembly for attachment to a bioreactor container holding a cell suspension. The bioreactor interface plate assembly comprising a bioreactor interface plate and a filter. The bioreactor interface plate including a port formed therein for egress of fluid from the bioreactor container. The filter attached to the bioreactor interface plate in fluid communication with the port, the filter positioned to be located within an internal volume of the bioreactor container while the bioreactor container is connected to the bioreactor interface plate. The filter configured to retain cells from the cell suspension in the internal volume when the fluid egresses from the bioreactor container via the port.

### CROSS-REFERENCE TO RELATED APPLICATIONS

This application is a national phase entry under 35 U.S.C. § 371 of International Patent Application PCT/GB2022/051967, filed Jul. 27, 2022, designating the United States of America and published as International Patent Publication WO 2023/007155 A1 on Feb. 2, 2023, which claims the benefit under Article 8 of the Patent Cooperation Treaty of United Kingdom Patent Application Serial No. 2111042.4, filed Jul. 30, 2021.

### TECHNICAL FIELD

The disclosure relates to a bioreactor interface plate for attachment to a bioreactor container holding a cell suspension.

### BACKGROUND

Biological handling processes, such as cell and gene therapy (CGT) manufacturing processes, are often complex and include manual steps across several devices. Equipment systems used in various steps or unit operations, of cell-based therapeutic products (CTP) manufacturing may include devices for various unit operations. The unit operations may include, for example, cell collection, cell isolation, selection, cell expansion, cell washing, volume reduction, cell storage or transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum, thus, there is a need for improved cell manufacturing equipment for various cell manufacturing procedures, for example, but not limited to stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags. Such bottles or flasks are widely used but suffer from several drawbacks. During cell culturing further culturing media may be added to the container, and some fluid may be extracted from the container, for example, a waste fluid.

### BRIEF SUMMARY

In accordance with one aspect of the present disclosure, there is provided a bioreactor interface plate for attachment to a bioreactor container holding a cell suspension. The bioreactor interface plate comprises a port for egress of fluid from the bioreactor container. The bioreactor interface plate further comprises a filter attached to the bioreactor interface plate so as to be located within an internal volume of the bioreactor container during use. The filter is in fluid communication with the port and configured to retain cells from the cell suspension in the internal volume when the fluid egresses from the bioreactor container via the port.

In examples, the bioreactor container is generally cylindrical and the bioreactor interface plate may be circular. The bioreactor interface plate may act as a lid or closure for the bioreactor container. The bioreactor interface plate may be substantially planar, i.e., flat. The bioreactor interface plate may have opposing major surfaces and one or more side surfaces.

In examples, the filter may comprise a filter housing attached to the bioreactor interface plate, and a filter member attached to filter housing. In examples, the filter member may be attached to the filter housing by welding, for example, hot plate welding or ultrasonic welding. The filter member may comprise a filter membrane or a depth filter.

In examples, the filter housing may be fixedly mounted to the bioreactor interface plate. For example, the filter housing may be mounted to the bioreactor interface plate by a threaded connection. In particular, the bioreactor interface plate may have a threaded spigot or a threaded hole, and the filter housing may have a corresponding threaded hole or threaded spigot. A fluid channel connecting the filter to the bioreactor interface plate, in particular, the port, may extend through the connection between the filter housing and the bioreactor interface plate.

In examples, the filter housing may be fixedly mounted to a central portion of the bioreactor interface plate such that the filter is disposed centrally within the bioreactor container. The central location of the filter may permit a filter with a larger surface area to be provided.

In examples, the filter housing may be suspended from the bioreactor interface plate by a flexible hose. In particular, a first end of the flexible hose may be connected to the bioreactor interface plate and in fluid communication with the port, and a second end of the flexible hose may be attached to the filter and adapted to receive fluid through the filter. The filter may be adapted to float on the surface of the fluid in the bioreactor container. The flexible hose allows the filter to move with the changing levels of fluid in the bioreactor container. Suction can be applied to the port to draw fluid from the bioreactor container, through the filter and flexible hose, to the port.

In examples, the filter is deployable. In particular, the filter housing may be suspended from the bioreactor interface plate by a flexible hose. The bioreactor interface plate may have a deployment mechanism arranged to hold the filter at or proximate to the bioreactor interface plate and operable to deploy the filter toward the fluid in the bioreactor container.

In examples, the bioreactor interface plate comprises first and second flexible hoses extending between the filter and the bioreactor interface plate. In such examples, the deployment mechanism may comprise an arm, in particular, a rotatable arm, arranged to engage the first and second flexible hoses. Rotation of the rotatable arm may deploy the filter. The rotatable arm may be rotatable to retract the filter.

In examples, the filter may comprise a cavity defined between an internal surface of the filter housing and the filter member. The port may be in fluid communication with the cavity. The filter may comprise a filter outlet in fluid communication with the port. The internal surface of the filter housing may be tapered. The internal surface of the filter housing may be tapered toward the filter outlet. The internal surface of the filter housing may be frustrum-shaped, for example, frustoconical. In examples, the filter further comprises spacer ribs disposed between the internal surface of the filter housing and the filter member. The spacer ribs act to hold the filter member away from the internal surface of the filter housing and so maintain the filter cavity.

In examples, the filter housing may further comprise an external surface facing the bioreactor interface plate. In examples, the external surface is frustrum-shaped with a convex surface facing the bioreactor interface plate. In particular, the external surface is frustoconical. Accordingly, fluid that flows onto the external surface will flow back into the container due to the incline of the external surface.

In examples, the port is adapted to connect to a fluid waste container to receive the fluid egressed from the bioreactor container. For example, the port may comprise a septum seal. The septum seal may connect to a fluid waste container via a needle that pierces the septum seal. The bioreactor interface plate may further comprise a connector interface associated with the port, the connector interface being adapted for connecting to the fluid waste container.

In other examples, the port may comprise a valve, cap or other closure that can be removed to provide a fluid connection to the fluid waste container.

In examples, the bioreactor interface plate may further comprise a fluid channel extending through the bioreactor interface plate between the filter and the port. In particular, the fluid channel may fluidly connect a filter outlet to the port. Accordingly, the port may be offset from the filter.

In accordance with another aspect of the present disclosure, a bioreactor interface plate for attachment to a bioreactor container holding a cell suspension. The bioreactor interface plate comprises a port for egress of fluid from the bioreactor container. The bioreactor interface plate further comprises a fluid channel extending through the bioreactor interface plate and connected to the port, the fluid channel having an outlet opening into the bioreactor container during use. In examples, a filter may be attached to the outlet as described above.

The fluid channel may be formed within the bioreactor interface plate and extend through the bioreactor interface plate in a direction parallel with major surfaces of the bioreactor interface plate. The fluid channel may be formed between two parts of the bioreactor interface plate that are joined together and define the fluid channel therebetween. In examples, the fluid channel comprises a radially extending portion extending from a filter outlet in a radial direction of the bioreactor interface plate, and a circumferentially extending portion extending from the radially extending portion in a circumferential direction of the bioreactor interface plate. The port may be located along the circumferentially extending portion.

In examples, the bioreactor interface plate may comprise a plurality of ports arranged along the fluid channel. In particular, the bioreactor interface plate may comprise a plurality of ports arranged along the circumferentially extending portion of the fluid channel. At least some, or all, of the plurality of ports may be for egress of fluid, and at least some, or all, of the plurality of ports, may be for ingress of fluid. The ports may be used either for egress or ingress of fluid.

In examples, a first port of the plurality of ports may be arranged at a first distance along the fluid channel from the filter outlet, and a second port of the plurality of ports may be arranged at a second distance along the fluid channel from the filter outlet. The first distance may be greater than the second distance. Accordingly, depending on which port is used, fluid may or may not flow through parts of the fluid channel previously used, and may or may not flow past a port that has been previously used. Ports can therefore be selected to improve sterility and to flush fluids out of the fluid channel.

In other examples, the fluid channel may comprise a hose or pipe extending between the filter outlet and the port. Accordingly, the port may be offset from the filter. In examples, the hose or pipe may be connected to more than one port, and a first port may be located between the filter outlet and a second port along the hose.

In some examples, the bioreactor interface plate may further comprise an opening through the bioreactor interface plate, and a septum arranged to seal the opening and permit a needle to pierce the septum for adding material to the bioreactor during use.

In examples, the filter member comprises a filter membrane. The filter membrane may comprise a polyester track etch (PETE) filter membrane, a polyethersulfone (PES) filter membrane, a polycarbonate track-etched (PCTE) membrane filter, a polyethylene membrane filter, or a polyvinylidene difluoride (PVDF) membrane filter. The filter membrane may be, for example, up to about 3 millimeters thick, preferably up to about 1.5 millimeters thick.

In other examples, the filter member comprises a depth filter. The depth filter may comprise a sintered polypropylene depth filter, a sintered ceramic depth filter, a sintered ultra-high molecular weight polyethylene (UHMWPE) depth filter, a sintered polytetrafluoroethylene (PTFE) depth filter, a glass fiber depth filter, or a sintered polypropylene fibers depth filter. The depth filter may have a thickness greater than about 1 millimeter, for example, a thickness greater than about 1.5 millimeters.

In examples, the bioreactor interface plate may further comprise an attachment member for attachment of the bioreactor interface plate to the bioreactor container such that the bioreactor container is sealed by the bioreactor interface plate and the filter is arranged in the internal volume of the bioreactor container.

In accordance with a further aspect of the present disclosure, there is provided a bioreactor comprising a bioreactor container defining an internal volume for holding a fluid cell suspension, and the bioreactor interface plate described above. The bioreactor interface plate is attached to the bioreactor container such that the bioreactor container is closed by the bioreactor interface plate and the filter is arranged in the internal volume of the bioreactor container.

In examples, the bioreactor container may comprise a base section and a side wall, and the bioreactor interface plate may be attached to the side wall opposite to the base section. Accordingly, the bioreactor interface plate may be a lid or closure for the bioreactor container.

The bioreactor container may be generally cylindrical, where the side wall is generally cylindrical and the base section is generally circular. The bioreactor interface plate may be generally circular and planar and adapted to be attached to an end of the bioreactor container opposite to the base section. The bioreactor interface plate and/or the bioreactor container may comprise a clamping element to attach the bioreactor interface plate to the bioreactor container. A seal may be provided between the bioreactor container and the bioreactor interface plate.

In examples, the side wall of the bioreactor container is a compressible and/or extendible side wall. In particular, the side wall may be a bellows wall. The side wall may include a plurality of inward folds and outward folds, interleaved with leaf segments. The inward and outward folds permit the leaf segments to fold against each other, thereby compressing the side wall, or vice versa to extend the side wall.

In accordance with a further aspect of the present disclosure, there is provided a method of extracting a fluid from a cell suspension held in the bioreactor container of the bioreactor described above. The method comprises at least partially submerging the filter in the cell suspension held in the bioreactor container. The method also comprises generating a pressure differential to move fluid through the filter and the outlet to a container.

In examples, at least partially submerging the filter in the cell suspension held in the bioreactor container may comprise moving or deploying the filter. For example, as described above, the filter can be deployed by a filter deployment mechanism.

In other examples, at least partially submerging the filter in the cell suspension held in the bioreactor container may comprise compressing the bioreactor container to raise a fluid level of the cell suspension within the bioreactor container. In particular, the bioreactor container may comprise a compressible wall, as described above, and the method may comprise compressing the bioreactor container to bring the fluid level up to the filter so that the filter is at least partially submerged.

In examples, generating a pressure differential to move the fluid through the filter and the port to a container comprises applying suction to the port to draw the fluid from the bioreactor container. Applying suction to the port may comprise expanding the container. For example, the container can be an expandable container and fluidly connected to the port. Expansion of the expandable container will generate suction to draw fluid from the bioreactor container. In some examples, the container comprises a syringe, and applying suction to the port may comprises extending the syringe.

In other examples, a suction pump may be used to apply suction to the port.

In examples, generating a pressure differential to move the fluid through the filter and the port to a container may additionally or alternatively comprise compressing the bioreactor to urge fluid through the filter and the port to the container.

In examples, the method may further comprise delivering a flushing fluid to the bioreactor container after extracting the fluid. The flushing fluid may be delivered to the bioreactor container through the filter. The flushing fluid may be delivered through the same port as the fluid was extracted through. Alternatively or additionally, the flushing fluid may be delivered through a different port than the fluid was extracted through. The flushing fluid passes through the filter and into the bioreactor container, washing cells from the filter.

In accordance with a further aspect of the present disclosure there is provided a filter for a bioreactor. The filter is positionable within an internal volume of the bioreactor. The filter comprises a filter housing comprising a connector for fluid connection to a port of the bioreactor. The filter also comprises a filter member attached to the filter housing.

In examples, the filter may comprise a cavity defined between an internal surface of the filter housing and the filter member. The filter may comprise a filter outlet in fluid communication with a bioreactor interface plate of the bioreactor. The internal surface of the filter housing may be tapered. The internal surface of the filter housing may be tapered toward the filter outlet. The internal surface of the filter housing may be frustrum-shaped, for example, frustoconical. In examples, the filter further comprises spacer ribs disposed between the internal surface of the filter housing and the filter member. The spacer ribs act to hold the filter member away from the internal surface of the filter housing and so maintain the filter cavity.

In examples, the filter housing may further comprise an external surface arranged to face the bioreactor interface plate. In examples, the external surface is frustrum-shaped with a convex surface facing the bioreactor interface plate. In particular, the external surface is frustoconical. Accordingly, fluid that flows onto the external surface will flow back into the container due to the incline of the external surface.

In examples, the filter member comprises a filter membrane. The filter membrane may comprise a polyester track etch (PETE) filter membrane, a polyethersulfone (PES) filter membrane, a polycarbonate track-etched (PCTE) membrane filter, a polyethylene membrane filter, or a polyvinylidene difluoride (PVDF) membrane filter. The filter membrane may be, for example, up to about 3 millimeters thick, preferably up to about 1.5 millimeters thick.

In other examples, the filter member comprises a depth filter. The depth filter may comprise a sintered polypropylene depth filter, a sintered ceramic depth filter, a sintered ultra-high molecular weight polyethylene (UHMWPE) depth filter, a sintered polytetrafluoroethylene (PTFE) depth filter, a glass fiber depth filter, or a sintered polypropylene fibers depth filter. The depth filter may have a thickness greater than about 1 millimeter, for example, a thickness greater than about 1.5 millimeters.

### BRIEF DESCRIPTION OF THE DRAWINGS

Example embodiments of the disclosure are now described, by way of example only, hereinafter with reference to the accompanying drawings, in which:

FIG. 1 illustrates a cell processing system including a bioreactor;

FIG. 2 illustrates a cell processing method;

FIG. 3 illustrates a bioreactor of the cell processing system of FIG. 1;

FIG. 4 illustrates a waste consumable for use with the bioreactor of FIG. 3;

FIG. 5 illustrates a connector for connecting the waste consumable of FIG. 4 to the bioreactor of FIG. 3;

FIG. 6 illustrates an example bioreactor with a filter;

FIG. 7 shows a detailed view of the bioreactor and filter of FIG. 6;

FIG. 8 shows the filter housing of the filter of FIGS. 6 and 7;

FIG. 9 shows the filter housing of the filter of FIGS. 6 to 8;

FIG. 10 shows another example bioreactor with a filter;

FIG. 11 shows the filter of FIG. 10;

FIG. 12 shows an example deployable filter;

FIG. 13 shows a deployment mechanism for the deployable filter of FIG. 12;

FIG. 14 illustrates an example interface plate of the bioreactor having a plurality of connector interfaces;

FIG. 15 illustrates a fluid channel and septum of the interface plate of FIG. 14;

FIG. 16 illustrates a further example interface plate of the bioreactor having a plurality of connector interfaces;

FIG. 17 illustrates a fluid channel and septum of the interface plate of FIG. 17, and

FIG. 18 illustrates a method of extracting fluid from the bioreactor and flushing the filter.

### DETAILED DESCRIPTION

The described example embodiments relate to an assembly for handling biological material. In particular, some embodiments relate to an assembly that is aseptic, or sterile. It is noted that the terms “aseptic” and “sterile” may be used interchangeably throughout the present disclosure. References to fluids in the detailed description are not intended to limit the scope of protection to such materials. As will be recognized by a person skilled in the art, fluids as described herein are merely an example of a suitable material for use with the assembly as described. Equally, reference may be made to a container, container, or the like, however, such references are not intended to limit the scope of protection to such containers or containers. As will be recognized by a person skilled in the art, containers, containers or the like are described herein as mere examples.

Certain terminology is used in the following description for convenience only and is not limiting. The words “upper” and “lower” designate directions in the drawings to which reference is made and are with respect to the described component when assembled and mounted. The words “inner,” “inwardly” and “outer,” and “outwardly” refer to directions toward and away from, respectively, a designated centerline or a geometric center of an element being described (e.g., a central axis), the particular meaning being readily apparent from the context of the description. Further, the terms “proximal” (i.e., nearer to) and “distal” (i.e., away from) designate positions relative to an axis or a point of attachment.

Further, as used herein, the terms “connected,” “affixed,” “coupled” and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first,” “second,” “third,” etc. merely indicate that different instances of like objects are being referred to and are not intended to imply that the objects so described must be in a given sequence, either temporally, spatially, in ranking or in any other manner. Like reference numerals are used to depict like features throughout.

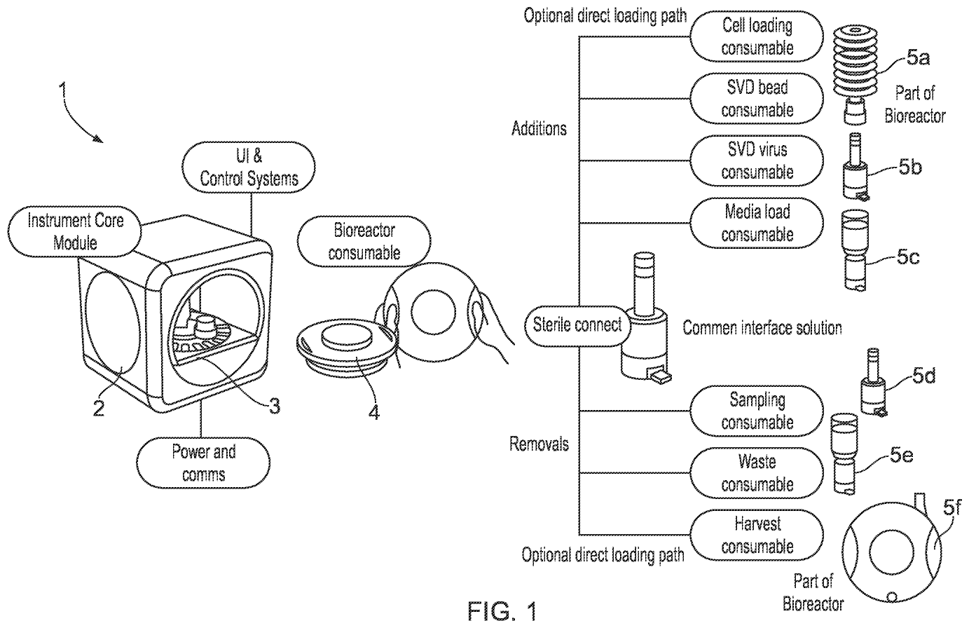

FIG. 1 shows a cell processing system 1 that includes a cell processing housing 2, a cell processing platform 3, a bioreactor 4, and various accessories, for example, “consumables” 5a-5f.

The cell processing housing 2 provides a closed environment for the cell processing platform 3 and is provided with power, connectivity and other utilities needed for the cell processing as described hereinafter. The cell processing platform 3 is adapted to receive the bioreactor 4 and support the bioreactor 4 within the cell processing housing 2. The cell processing platform 3 may include various components and systems that interact with the bioreactor 4 and/or the consumables 5a-5f. For example, the cell processing platform 3 may include an agitator that acts to agitate the bioreactor 4 so as to agitate a cell suspension provided within the bioreactor 4. In other examples, the cell processing platform 3 may include an accessory support arm adapted to hold one or more consumables 5a-5f. In examples, the cell processing platform 3 may include an actuator operable to actuate one or more the consumables 5a-5f. The cell processing platform 3 may be configured for automated operation of the cell processing system 1, or may permit manual operation.

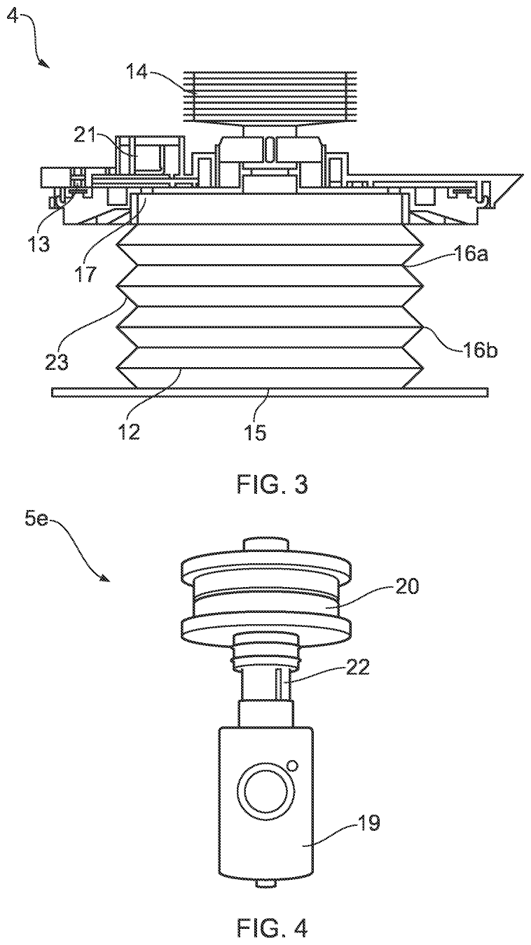

The bioreactor 4, described in more detail with reference to FIG. 3, includes a container 12 and an interface plate 13. During use the container 12 holds a fluid in which the cell processing occurs. In particular, the fluid comprises a population of cells present in a liquid medium. The container 12 may be expandable, for example, by having a bellows wall. The bioreactor 4 is held in the cell processing housing 2 such that the container 12 can expand and retract as it is filled and emptied. The interface plate 13 may be engaged by the cell processing platform 3 and provides various functions relating to the bioreactor 4. For example, the interface plate 13 may have one or more connectors for transfer of fluids into and out of the container 12. The bioreactor 4 also has a filter for filtering cells from the fluid as the fluid egresses from the container 12. In this way, fluid can be removed from the container 12 and the cells retained in the container 12.

The consumables 5a-5f are for connecting to the bioreactor 4, optionally via the cell processing platform 3, in order to facilitate process steps of the cell culturing process.

In examples, the consumables may include a cell delivery consumable 5a for delivering cells to the container 12. In examples, the consumables may include a fluid delivery consumable 5b, for example, a particle suspension delivery consumable or virus suspension delivery consumable. The particle suspension may comprise magnetic particles, for example, magnetic beads. In examples, the consumables may include a media delivery consumable 5c for delivering a media, in particular, a cell culturing media, to the container 12. In examples, the consumables may include a sampling consumable 5d, for example, a vial or vacutainer. In examples, the consumables may include a waste consumable 5e for receiving waste fluid from the container 12. In examples, the consumables may include a cell harvesting consumable 5f for receiving cells from the container 12 at the end of a cell culturing process.

In various examples, the population of cells provided to the container 12 may comprise any human or animal cell type, for example: any type of adult stem cell or primary cell, T cells, CAR-T cells, monocytes, leukocytes, erythrocytes, NK cells, gamma delta t cells, tumor infiltrating t cells, mesenchymal stem cells, embryonic stem cells, induced pluripotent stem cells, adipose derived stem cells, Chinese hamster ovary cells, NS0 mouse myeloma cells, HELA cells, fibroblasts, HEK cells, insect cells, organoids, etc. Suitably the population of cells may comprise T-cells. Alternatively, the population of cells may comprise any microorganism cell type, for example: bacterial, fungal, Archaean, protozoan, algal cells.

In examples, the cell culturing medium may be any sterile liquid capable of maintaining cells. The cell culturing medium may be selected from: saline or may be a cell culture medium. The cell culturing medium may be selected from any suitable medium, for example: DMEM, XVIVO 15, TexMACS. The cell culturing medium may be appropriate for the type of cells present in the population. For example, the population of cells comprises T cells and the cell culturing medium comprises XVIVO 10. In examples, the cell culturing medium may further comprise additives, for example: growth factors, nutrients, buffers, minerals, stimulants, stabilizers or the like.

In examples, the cell culturing medium comprises growth factors such as cytokines and/or chemokines. The growth factors may be appropriate for the type of cells present in the population and the desired process to be carried out. The cell culturing medium may comprise stimulants such as antigens or antibodies, which may be mounted on a support. Suitable stimulants are appropriate for the type of cells present in the population and the desired process to be carried out. When culturing T-cells, for example, antibodies are provided as a stimulant in the cell culturing medium. The antibodies may be mounted on an inert support such as beads, for example: dynabeads. The additives may be present in the liquid medium at an effective concentration. An effective concentration can be determined by the skilled person on the basis of the population of cells and the desired process to be carried out using known teachings and techniques in the art. In examples, the population of cells are seeded in the cell culturing medium at a concentration of between 1×104 cfu/ml up to 1×108 cfu/ml.

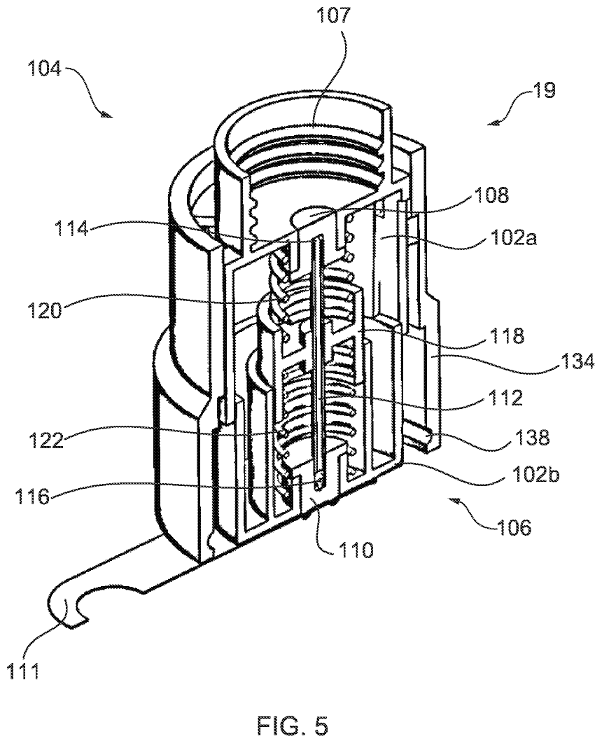

In examples, each of the consumables 5a-5f is connectable to the bioreactor 4 by a common connector. The connector may be that described in co-pending patent application PCT/GB2020/053229, as described further with reference to FIG. 5. The connector is connectable to the consumable and to the interface plate 13, and then the connector is actuatable to drive a needle through seals in the interface plate 13 and the consumable and create a fluid path therebetween. Accordingly, the connector can be used to attach a consumable to the interface plate 13 and create a fluid connection for transfer of fluids from the consumable into the container 12, or vice versa.

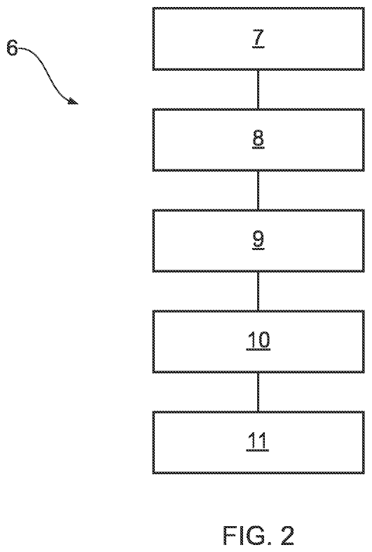

FIG. 2 schematically illustrates a cell culturing process 6 based on the cell processing system 1 described with reference to FIG. 1. As shown in FIG. 2, initially the consumables 5a-5f are prepared 7. For example, a cell delivery consumable 5a may be filled with a cell suspension, and a particle loading consumable 5b may be filled with particles such as magnetic beads. A connector may be attached to the consumable 5a-5f before or after preparation. Preparation of the consumable(s) 5a-5f may include unpackaging the consumable(s) 5a-5f from a sterile package. It will be appreciated that only the consumables 5a-5f needed for the particular process, and the particular stage of the process, are prepared. For example, some processes would not use magnetic particles so a particle loading consumable 5b is not needed, and the cell harvesting consumable 5f is only needed at the end of the process 6.

Next, cells are loaded into the bioreactor 4, 8. In particular, a cell delivery consumable 5a is connected to the bioreactor 4 and operated to transfer a cell suspension from the cell delivery consumable 5a into the bioreactor 4. The cell delivery consumable 5a is connected to the bioreactor 4 via a connector, as described above, which forms a fluid connection between the cell delivery consumable 5a and the bioreactor 4.

Either before or after loading cells into the bioreactor 4, 8, the bioreactor 4 is loaded into the cell processing housing 2, 9. In some examples, the bioreactor 4 is attached to the cell processing platform 3 within the cell processing housing 2.

Within the cell processing housing 2 the cells are processed 10 in the bioreactor 4. During cell processing 10 the pressure, temperature, pH and other environmental characteristics within the bioreactor 4 are controlled to ensure that conditions enable cell processing. Cell processing 10 may comprise reprogramming the cells, for example, by using CAR-coding viral DNA. Cell processing 10 may comprise cell culturing.

During cell processing 10 additional consumables 5a-5f may be used to add materials to the bioreactor 4, to extract a sample from the bioreactor 4, and/or to extract waste from the bioreactor 4. For example, a delivery consumable 5b may be used to add magnetic beads to the bioreactor. In examples, a delivery consumable 5b may be used to add a virus suspension or solution to the bioreactor (e.g., CAR-coding viral DNA). In examples, a media loading consumable 5c may be used to add one or more media to the bioreactor 4. For example, a media loading consumable 5c may be used to add a balanced salt solution or a basal media to the bioreactor 4. In examples, a sampling consumable 5d may be used to extract a sample from the bioreactor for testing.

In examples, during or after cell processing 10 a waste consumable 5e may be used to extract a waste media from the bioreactor 4. As explained in detail hereinafter, the waste consumable 5e is attachable to the interface plate 13 and a fluid connection is established between the waste consumable 5e and the container 12. Fluid can then be extracted from the container 12 into the waste consumable 5e. The fluid passes through a filter that retains cells in the container 12.

After cell processing 10 the cells are harvested 11. Cell harvesting 11 may initially use a waste consumable 5e to extract a waste component. A harvesting consumable 5f can be attached to bioreactor 4 to receive the cells from the bioreactor 4. The cells may be harvested in a media, for example, a cell suspension may be harvested.

As shown in FIG. 3, the bioreactor 4 comprises a container 12 and an interface plate 13. The interface plate 13 comprises at least one connector interface 21 for connecting to an external component, for example, one of the consumables 5a-5f described above. Each connector interface 21 may include a port. In examples, the port of each connector interface 21 includes a septum seal that maintains a sealed environment within the container 12 and also permits a needle to pass through to create a fluid connection into the container 12. In other examples, each port may have a valve, a cap, or other closure that provides an openable or breakable seal.

The container 12 is a compressible container. In particular, the container 12 has a bottom wall 15 disposed opposite to the interface plate 13, and a compressible wall 23 defining a sidewall of the container 12. A top part 17 of the compressible wall 23 is attached to the interface plate 13. The top part 17 may include a rigid ring or similar for attaching to the interface plate 13. The compressible wall 23 is compressible such that the bottom wall 15 can move toward and away from the interface plate 13, changing the internal volume of the container 12.

The compressible wall 23 may be a bellows wall, having a concertina arrangement that allows the compressible wall 23 to fold onto itself in order to compress. In particular, the compressible wall 23 may comprise a series of alternately arranged inward folds 16a and outward folds 16b that allow the compressible wall 23 to compressible like a bellows or concertina. Leaf portions extend between the inward folds 16a and the outward folds 16b. The inward folds 16a and outward folds 16b may be formed by thinned sections in the compressible wall 23, with the inward folds 16a comprise a thinned section arranged on the outer surface of the compressible wall 23, and the outward folds 16b comprising a thinned section arranged on the inner surface of the compressible wall 23.

The container 12 can therefore expand and contract, or be expanded and contracted, according to the material held in the container 12. In particular, the compressible container 12 may expand as the cell culture within the container 12 grows, and/or as additional materials are added. The cell processing housing (2, see FIG. 1) may comprise an actuator adapted to move, for example, push and/or pull, the bottom wall 15 of the container 12 and/or the interface plate 13 to change the volume of the container 12.

As illustrated, the interface plate 13 also includes an expansion container 14, otherwise called a breathing container. The expansion container 14 allows for the container 12 to expand and contract without greatly changing the pressure in the container 12. Alternatively or additionally, the expansion container 14 may be operable, for example, by being mechanically or manually compressed or expanded, to expand or retract the compressible wall 23 of the container 12 and thereby change a volume of the container 12. Alternatively or additionally, the expansion container 14 may be operable, for example, by being mechanically or manually compressed or expanded, to alter the pressure within the container 12.

FIG. 4 illustrates a waste consumable 5e. As described above, the waste consumable 5e is connectable to the bioreactor 4 via the interface plate 13, in particular, via the connector interface 21. As mentioned above, the connector interface 21 may include a port, for example, a septum seal, for providing a fluid connection between the container 12 and the waste consumable 5e.

The waste consumable 5e has a connector 19, described in more detail with reference to FIG. 5, that connects to the connector interface 21 of the interface plate 13 and forms a fluid connection between the waste consumable 5e and the container 12, via the port. The waste consumable 5e also has an expandable container 20 in fluid communication with the connector 19 via an intermediate portion 22. In some examples, the waste consumable 5e alternatively comprises a syringe arrangement having a plunger that can be withdrawn to draw fluid into a container of the syringe.

In FIG. 4 the expandable container 20 is shown in a compressed state, and as fluid is extracted through the connector 19 and into the expandable container 20 the expandable container 20 will expand. In various examples described hereinafter, the expandable container 20 may be actuated to expand and thereby generate suction to draw the fluid into the expandable container 20.

The expandable container 20 may have a bellows wall similar to the compressible wall 23 of the container 12. In other examples the expandable container 20 may comprise a telescopically expandable container, or an elastically expandable container.

FIG. 5 illustrates the connector 19. The connector 19 is used to attach a consumable 5a-5f to the bioreactor 4, in particular, to the connector interface 21 of the interface plate 13 of the bioreactor 4. The connector 19 may be as described in patent application PCT/GB2020/053229.

In particular, as shown in FIG. 5 the connector 19 comprises a housing 102 having an upper housing portion 102a and a lower housing portion 102b. The housing 102 extends along a longitudinal axis between a distal end 104 and a proximal end 106. The upper housing portion 102a may be axially moveable, or slidable, with respect to the lower housing portion 102b, as will be described further below.

The housing 102 includes a threaded portion 107 at its distal end 104 for connecting to a corresponding threaded portion of the intermediate portion (22, see FIG. 4) of the waste consumable (5e, see FIG. 4). The threaded portion 107 is formed on the upper housing portion 102a. As will be clear to the skilled person, the housing 102 may be provided without the threaded portion 107, and instead be provided with another suitable connection mechanism for connecting to a portion of the waste consumable (5e, see FIG. 4).

The connector 19 also includes a connector portion at its proximal end 106 for connecting to the bioreactor (4, see FIG. 3), in particular, a connector interface (21, see FIG. 4B) of the bioreactor (4, see FIG. 3). The connector portion may be a groove 138, as illustrated in FIG. 5, configured to receive one or more protrusions or legs on the bioreactor. Alternatively, the connector 19 may comprise a threaded portion or other connector portion for connecting to the bioreactor. In some examples, the connector 19 is held or pressed against the connector interface (21, see FIG. 4B) of the bioreactor (4, see FIG. 3) and does not connect.

In this embodiment, the connector 19 includes a first septum seal 108 disposed at the distal end 104 of the housing 102, and a second septum seal 110 disposed at the proximal end 106 of the housing 102. The first septum seal 108 includes a substantially planar, i.e., flat, pierceable surface facing outwardly at the distal end 104. The second septum seal 110 includes a generally annular portion, extending outwardly at the proximal end 106, enclosing a substantially planar, i.e., flat, pierceable surface facing outwardly at the proximal end 106. The housing 102 further includes a hollow needle 112 that is biasedly mounted within the housing 102. The hollow needle 112 is generally coaxially aligned with the longitudinal axis. The hollow needle 112 includes a first end 114, facing the first septum seal 108, and a second end 116, facing the second septum seal 110. The first end 114 is configured to be able to pierce the first septum seal 108, in use, and the second end 116 is configured to be able to pierce the second septum seal 110, in use. The first septum seal 108, the second septum seal 110, or both the first and second septum seal 108, 110 may optionally be provided with a removable aseptic paper seal 111.

The hollow needle 112 is mounted within the housing 102 through a collar 118 that is spring-biased by a first spring 120 and a second spring 122. In other embodiments, the hollow needle 112 may be mounted in another suitable manner, for example, the hollow needle 112 may be statically mounted, i.e., such that it does not move, and the housing 102 may be moveable about the hollow needle 112. The first spring 120 acts between the distal end 104 of the housing 102 and the collar 118. The second spring 122 acts between the proximal end 106 of the housing 102 and the collar 118. In this way, the first spring 120 provides a first biasing force to the hollow needle 112, via the collar 118, in a direction toward the proximal end 106 of the housing 102, and the second spring 122 provides a second biasing force to the hollow needle 112, via the collar 118, in a direction toward the distal end 104 of the housing 102.

The connector 19 further includes an actuating mechanism for causing the hollow needle 112 to pierce the septum seals 108, 110. By piercing the first and second septum seals 108, 110 the hollow needle 112 creates a fluid path between the distal end 104 and the proximal end 106 of the connector 19, and so during use creates a fluid connection between the waste consumable 5e and the container 12 of the bioreactor 4.

In the example illustrated in FIG. 5 the actuating mechanism includes an outer sleeve 134 that is arranged to collapse the upper housing portion 102a with respect to the lower housing portion 102b. The outer sleeve 134 is rotatable with respect to the housing 102 about the central longitudinal axis of the housing 102. For example, one of the outer sleeve 134 and the housing 102 may include a helical groove, and the other of the outer sleeve 134 and housing 102 may include a protrusion that engages the groove such that when the upper housing portion 102a collapses with respect to the lower housing portion 102b the outer sleeve 134 is rotated.

When the connector 19 is attached to the waste consumable (5e, see FIG. 4), for example, via the threaded portion 107, the first septum seal 108 seals the end of the waste consumable (5e, see FIG. 4). The proximal end 106 of the connector 19 is then attached to the connector interface (21, see FIG. 4), for example, by a clipping mechanism, a sliding mechanism, a threaded connection, or clamping. In this position, actuation of the actuating mechanism, in particular, rotation of the outer sleeve 134, causes the upper housing portion 102a to collapse with respect to the lower housing portion 102b and the hollow needle 112 pierces the first septum seal 108 and the second septum seal 110 and creates a fluid connection through the connector 19, between the waste consumable (5e, see FIG. 4) and the bioreactor (4, see FIG. 4).

Accordingly, the connector 19 initially provides a sealing closure for the waste consumable (5e, see FIG. 4), and the fluid connection is formed entirely within the connector 19, which advantageously maintains a sterile environment.

Once fluid has been transferred from the bioreactor (4, see FIG. 3) into the waste consumable 5e the actuation mechanism can be reversed so that the hollow needle 112 withdraws from the first septum seal 108 and optionally also the second septum seal 110. The first and/or second septum seal 108, 110 reseal on withdrawal of the hollow needle 112. The connector 19, and the waste consumable (5e, see FIG. 4), can then be detached from the bioreactor (4, see FIG. 3).

In examples, an end of the waste consumable 5e, in particular, an end of the intermediate portion 22 illustrated in FIG. 4, may comprise a plug seal, for example, a septum seal, so that the waste consumable 5e is sealed before the connector 19 is connected thereto. The plug seal of the intermediate portion 22 can be pierced by the hollow needle 112 when the connector 19 is actuated.

In examples, the connector interface 21 of the bioreactor 4 illustrated in FIG. 3 comprises a further septum seal that is pierced by the hollow needle 112 in use. The further septum seal forms a port in the connector interface 21. Accordingly, when the connector 19 is detached the bioreactor 4 remains sealed.

In the examples described hereafter the bioreactor 4 comprises a filter 24. The filter 24 is arranged in a fluid path between the internal volume of the container 12 and the connector interface 21 of the interface plate 13. In this way, fluid is filtered by the filter 24 as it egresses from the container 12. In particular, the filter 24 is configured to filter cells from the fluid, such that the cells are retained in the bioreactor 4 and other fluid, in particular, waste media, is extracted from the bioreactor 4. Accordingly, waste fluid can be removed from the bioreactor 4 while cells are retained in the bioreactor 4. Such a fluid wasting process may be performed one or more times during cell culturing to remove exhausted media and/or to increase the concentration of cells. Waste media may be removed from the bioreactor 4 before the cells are harvested at the end of the cell culturing process.

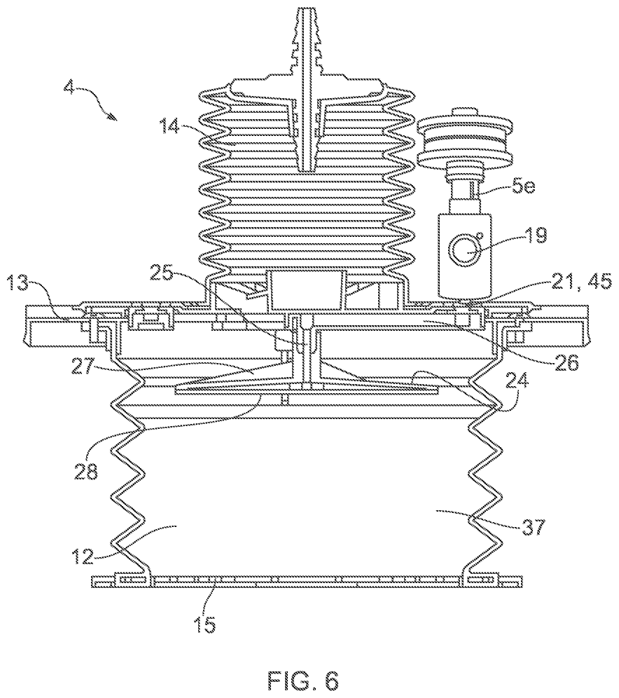

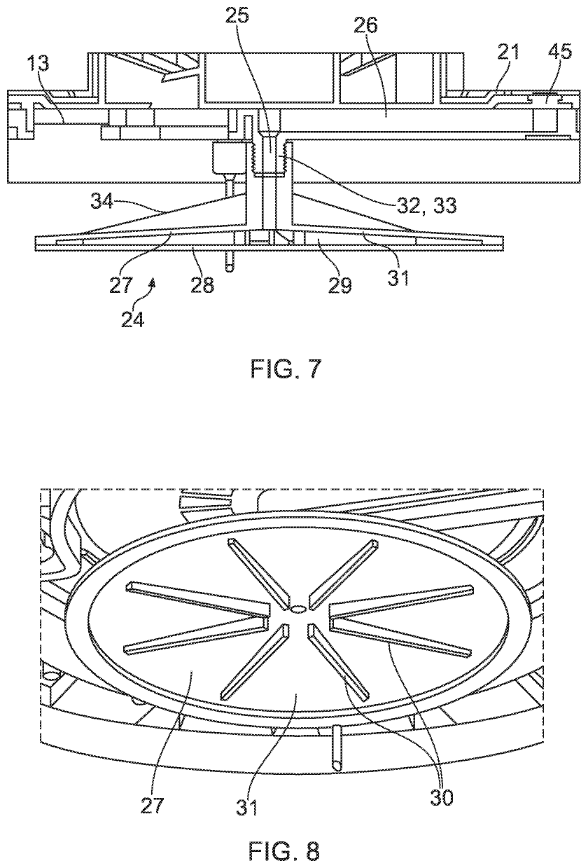

In the example of FIGS. 6 to 8 the bioreactor 4 comprises a filter 24. As shown more clearly in FIGS. 7 and 8, the filter 24 has a filter housing 27 and a filter member 28. The filter member 28 may be a filter membrane or a depth filter. A filter cavity 29 is defined between the filter housing 27 and the filter member 28. The filter cavity 29 is in fluid communication with a filter outlet 25.

As shown in FIG. 8, one or more spacer ribs 30 may be provided on the filter housing 27 and extend into the filter cavity 29. The spacer ribs 30 hold the filter member 28 away from an internal surface 31 of the filter housing 27 and ensure that the filter cavity 29 is maintained. In alternative examples the filter member 28 may have one or more protrusions that engage the internal surface 31 of the filter housing 27 to maintain the filter cavity 29.

As illustrated, the filter 24 is attached to the interface plate 13 and the filter outlet 25 is in fluid communication with a port 45 of the connector interface 21. The filter outlet 25 is in fluid communication with the port 45 through a fluid channel 26 formed in the interface plate 13. The fluid channel 26 extends in a sideways or radial direction within the interface plate 13 so that the port 45 is offset from the filter outlet 25. The interface plate 13 may comprise a plurality of connector interfaces 21 having a plurality of ports 45, and the fluid channel 26 may be connected to one or more of the plurality ports 45.

The filter housing 27 is attached to the interface plate 13. In particular, the filter housing 27 comprises a threaded hole 32 and the interface plate 13 comprises a corresponding threaded spigot 33 that is screwed into the threaded hole 32 to attach the filter housing 27 to the interface plate 13. The threaded spigot 33 has a fluid channel in communication with the fluid channel 26.

It will be appreciated that in other examples the filter housing 27 may be connected to the interface plate 13 by another connection, for example, the filter housing 27 may have an external thread that engages an internal thread of the interface plate 13.

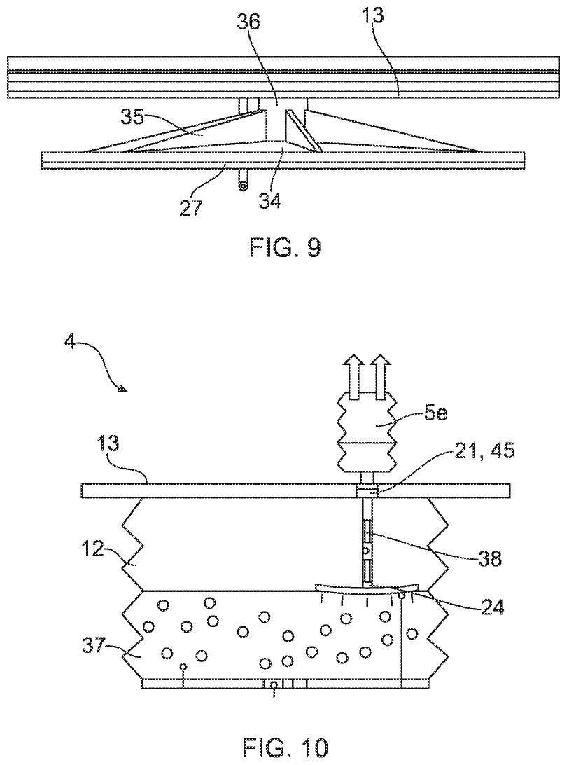

As shown in FIG. 7, an upper surface 34 of the filter housing 27 may be frustrum-shaped, in particular, frustoconical, with a convex surface directed upwards toward the interface plate 13. In other words, the upper surface 34 of the filter housing 27 is inclined toward the container 12. Accordingly, any fluid that flows onto the upper surface 34 will flow back into the container 12. Additionally, as shown in FIG. 9, the filter housing 27 may comprise one or more ribs 35 extending from the upper surface 34 and connecting the upper surface 34 to a central hub portion 36 of the filter housing 27 where the filter outlet 25 is formed.

As shown in FIGS. 6 to 8, the filter outlet 25 is central relative to the filter member 28. This can help to ensure that the fluid flows through the filter member 28 at a substantially even rate across the surface of the filter member 28, and can help to prevent blockages in the filter member 28. In addition, the filter 24 is attached to the interface plate 13 at a central position of the container 12. Accordingly, the filter 24, in particular, the filter member 28, can be of increased size compared to an offset mounting position. In examples, the filter member 28 may be sized to cover a majority of the cross-sectional area of the container 12, for example, greater than 50% or greater than 75% of the cross-sectional area of the container 12.

In examples, the filter member 28 comprises a filter membrane, for example, a polyester track etch (PETE) filter membrane, a polyethersulfone (PES) filter membrane, a polycarbonate track-etched (PCTE) membrane filter, a polyethylene membrane filter, or a polyvinylidene difluoride (PVDF) membrane filter. The filter membrane may be, for example, up to about 3 millimeters thick, preferably up to about 1.5 millimeters thick. In other examples, the filter member 28 comprises a depth filter, for example, a sintered polypropylene depth filter, a sintered ceramic depth filter, a sintered ultra-high molecular weight polyethylene (UHMWPE) depth filter, a sintered polytetrafluoroethylene (PTFE) depth filter, a glass fiber depth filter, or a sintered polypropylene fibers depth filter. The depth filter may have a thickness greater than about 1 millimeter, for example, a thickness greater than about 1.5 millimeters.

In examples, the filter member 28 is attached to the filter housing 27 by welding, for example, hot plate welding or ultrasonic welding. Alternatively, the filter member 28 may be attached to the filter housing 27 by a clip or clamp. A seal, for example, a gasket or O-ring, may be provided between the filter member 28 and the filter housing 27.

Accordingly, fluid can egress from the container 12, through the filter member 28, into the filter cavity 29, through the filter outlet 25, and through the fluid channel 26 to the port 45 of the connector interface 21. A waste consumable 5e, for example, as shown in FIG. 4, can be connected to the connector interface 21 to receive the fluid via the port 45. The filter member 28 retains cells in the container 12.

Referring to FIG. 6, fluid can be extracted from the container 12 by attaching a waste consumable 5e to the connector interface 21 of the interface plate 13. The waste consumable 5e forms a fluid connection with the fluid channel 26 via the port 45.

In one example, the container 12 can be compressed, in particular, by moving the bottom wall 15 of the container 12 toward the interface plate 13, or by moving the interface plate 13 toward the bottom wall 15. The container 12 is compressed until the fluid 37 in the container 12 reaches the filter 24. Once the filter 24 is at least partially submerged in the fluid 37 the waste consumable 5e is expanded (or if a syringe, plunger is withdrawn) to generate suction that draws the fluid through the filter 24 and the fluid channel 26 and into the waste consumable 5e.

In an alternative example, the container 12 is compressed to pressurize the fluid 37 within the container 12 and drive the fluid through the filter 24 and the fluid channel 26 and into the waste consumable 5e. In this example, other inlets and outlets through the interface plate 13, including the expansion container 14, are closed or shut such that fluid only egresses to the waste consumable 5e.

In another example, fluid can be egressed into the waste consumable 5e by a combination of expanding the waste consumable 5e (or if a syringe, withdrawing the plunger) and compressing the container 12.

FIG. 10 illustrates an alternative example filter 24 within the container 12 of the bioreactor 4. In this example, the filter 24 is connected to the interface plate 13 via a flexible hose 38. The filter 24 is adapted to float on the fluid 37 in the container 12. The filter 24 is suspended from the interface plate 13 by the flexible hose 38. The flexible hose 38 is fluidly connected to a waste consumable 5e through a connector interface 21 and port 45 in the connector interface 21. As illustrated in FIG. 10, expanding the waste consumable 5e generates suction in the flexible hose 38 that draws fluid through the filter 24 and into the waste consumable 5e. Cells are retained in the container 12 by the filter 24.

The flexible hose 38 permits the filter 24 to float on the fluid 37 regardless of how much fluid 37 is in the container 12, ensuring that fluid can be extracted when the fluid level changes.

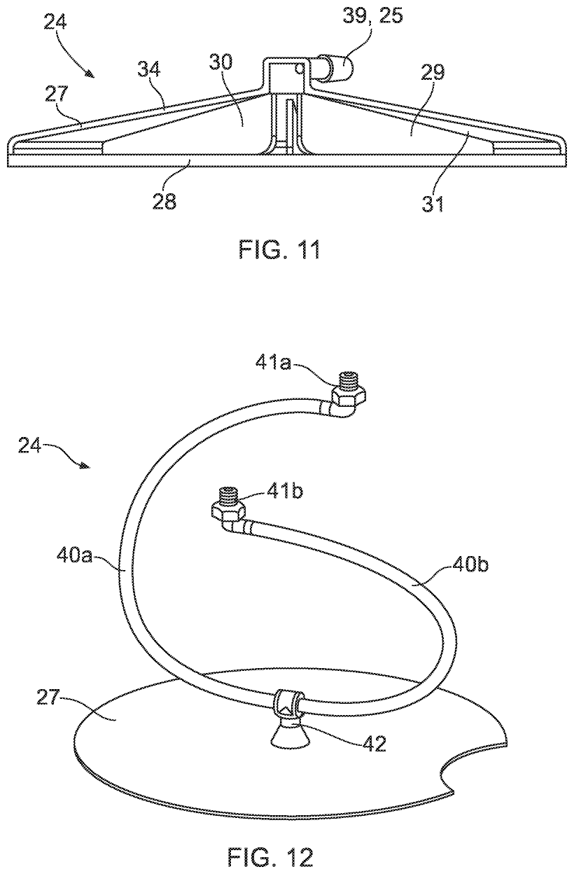

FIG. 11 shows the filter 24 of FIG. 10 in more detail. The filter 24 comprises a filter housing 27 and filter member 28, for example, a filter membrane or depth filter. A filter cavity 29 is defined in the filter 24, between the filter housing 27 and the filter member 28. The filter housing 27 has a filter outlet 25 defined in a hose connector 39 that is connected to the flexible hose (38, see FIG. 10).

The filter housing 27 comprises a one or more spacer ribs 30 extending into the filter cavity 29. The spacer ribs 30 hold the filter member 28 away from an internal surface 31 of the filter housing 27 and ensure that the filter cavity 29 is maintained. In alternative examples the filter member 28 may have one or more protrusions that engage the internal surface 31 of the filter housing 27 to maintain the filter cavity 29.

As illustrated, an upper surface 34 of the filter housing 27 may be frustrum-shaped, in particular, frustoconical, and convex in an upward direction toward the interface plate 13. In other words, the upper surface 34 of the filter housing 27 may be inclined toward the container (12, see FIG. 6). Accordingly, any fluid that flows onto the upper surface 34 will flow back into the container.

The filter 24, in particular, the filter housing 27 and filter member 28, are buoyant within the fluid 37 in the container 12. The buoyancy may be provided by the materials of the filter 24 and/or by air in the filter cavity 29. For example, the filter housing 27 may comprise a high density polyethylene (HDPE).

In examples, the filter member 28 comprises a filter membrane, for example, a polyester track etch (PETE) filter membrane, a polyethersulfone (PES) filter membrane, a polycarbonate track-etched (PCTE) membrane filter, a polyethylene membrane filter, or a polyvinylidene difluoride (PVDF) membrane filter. The filter membrane may be, for example, up to about 3 millimeters thick, preferably up to about 1.5 millimeters thick. In other examples, the filter member 28 comprises a depth filter, for example, a sintered polypropylene depth filter, a sintered ceramic depth filter, a sintered ultra-high molecular weight polyethylene (UHMWPE) depth filter, a sintered polytetrafluoroethylene (PTFE) depth filter, a glass fiber depth filter, or a sintered polypropylene fibers depth filter. The depth filter may have a thickness greater than about 1 millimeter, for example, a thickness greater than about 1.5 millimeters.

The filter member 28 is attached to the filter housing 27, for example, by welding such as hot plate welding or ultrasonic welding.

FIG. 12 illustrates an alternative example filter 24 for the example bioreactor 4 illustrated in FIG. 10. In this example, the filter 24 is connected to the interface plate 13 via a first flexible hose 40a and a second flexible hose 40b. The filter 24 is as described with reference to FIG. 11, with a filter housing 27 and a filter member 28 defining a filter cavity 29. A filter outlet 42 of the filter 24 is connected to both of the first flexible hose 40a and the second flexible hose 40b, for example, using a T-fitting or a Y-fitting. The filter 24 is adapted to float on the fluid 37 in the container 12 as shown in FIG. 10.

The first and second flexible hoses 40a, 40b are fluidly connected to a waste consumable 5e through the connector interface 21 in a manner similar to as shown in FIG. 10. In this example, the first and second flexible hoses 40a, 40b are connected to the interface plate 13 by respective first and second connectors 41a, 41b. The first and second connectors 41a, 41b may be connected to the interface plate 13 at different locations. The first and second connectors 41a, 41b are fluidly connected to the port 45 of the connector interface 21 via a fluid channel in the interface plate 13. The first and second connectors 41a, 41b are connected to the fluid channel at different locations along the fluid channel.

As illustrated in FIG. 10, expanding the waste consumable 5e generates suction in the first and second flexible hoses 40a, 40b that draws fluid through the filter 24 and into the waste consumable 5e. Cells are retained in the container 12 by the filter 24.

The first and second flexible hoses 40a, 40b permit the filter 24 to float on the fluid 37 regardless of how much fluid 37 is in the container 12, ensuring that fluid can be extracted when the fluid level changes.

The filter 24, in particular, the filter housing 27 and filter member 28, are buoyant within the fluid 37 in the container 12. The buoyancy may be provided by the materials of the filter 24 and/or by air in the filter cavity 29. For example, the filter housing 27 may comprise a high density polyethylene (HDPE).

In examples, the filter member 28 comprises a filter membrane, for example, a polyester track etch (PETE) filter membrane, a polyethersulfone (PES) filter membrane, a polycarbonate track-etched (PCTE) membrane filter, a polyethylene membrane filter, or a polyvinylidene difluoride (PVDF) membrane filter. The filter membrane may be, for example, up to about 3 millimeters thick, preferably up to about 1.5 millimeters thick. In other examples, the filter member 28 comprises a depth filter, for example, a sintered polypropylene depth filter, a sintered ceramic depth filter, a sintered ultra-high molecular weight polyethylene (UHMWPE) depth filter, a sintered polytetrafluoroethylene (PTFE) depth filter, a glass fiber depth filter, or a sintered polypropylene fibers depth filter. The depth filter may have a thickness greater than about 1 millimeter, for example, a thickness greater than about 1.5 millimeters.

The filter member 28 is attached to the filter housing 27, for example, by welding such as hot plate welding or ultrasonic welding.

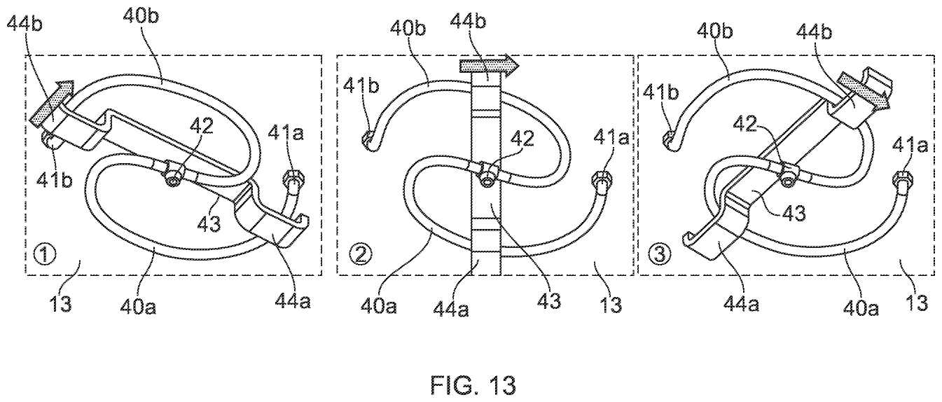

FIG. 13 illustrates a mechanism for deploying and retracting the filter 24 of FIG. 12. As illustrated, the interface plate 13 comprises a rotatable arm 43 having a first engaging portion 44a and a second engaging portion 44b. The first and second engaging portions 44a, 44b each comprise a bend shaped to retain the first and second flexible hoses 40a, 40b, respectively, between the rotatable arm 43 and the interface plate 13.

In the position shown in the left-most image of FIG. 13 the first and second engaging portions 44a, 44b are proximal to the first and second connectors 41a 41b and the full lengths of the first and second flexible hoses 40a, 40b are free to permit the filter 24 to be deployed into the container 12 to its maximum extent.

The middle and right-most images of FIG. 13 show that rotation of the rotatable arm 43 pulls the flexible hoses 40a, 40b toward and against the interface plate 13, reducing the proportion of the flexible hoses 40a, 40b that is free, and thereby pulling the filter outlet 42 and the filter 24 toward the interface plate 13. Accordingly, rotation of the rotatable arm 43 acts to deploy and retract the filter 24 within the container 12.

In the example of FIG. 13 the filter 24 can be deployed (i.e., lowered to float on the fluid within the container) when a waste extraction process is performed. When not in use the filter 24 can be retracted against the interface plate 13.

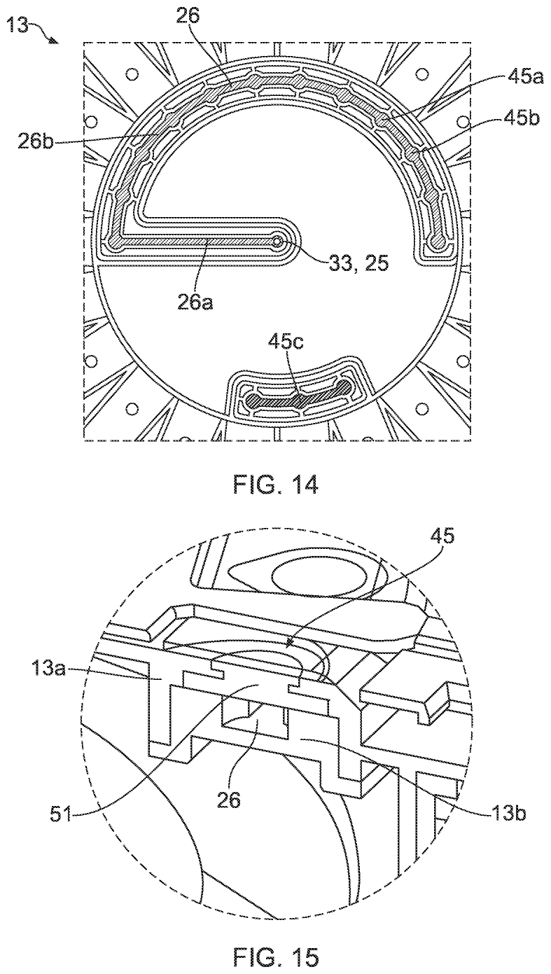

FIG. 14 illustrates the fluid channel 26 in the interface plate 13, and the locations of the ports 45a, 45b along the fluid channel 26. As described previously, each port 45 may be associated with a connector interface (21, see FIG. 4) for connection to a consumable, such as a waste consumable (5e, see FIG. 4). As illustrated, the interface plate 13 includes the threaded spigot 33 for connection to the filter 24 as shown in FIG. 7, although the example filters 24 of FIGS. 11 to 13 may be connected to the threaded spigot 33 instead. As shown in FIG. 6, the filter outlet 25 is located at threaded spigot 33.

The fluid channel 26 extends from the threaded spigot 33 and therefore from the filter outlet 25. The fluid channel 26 has a radially extending hose 26a extending from the filter outlet 25 toward an edge of the interface plate 13. The fluid channel 26 also has a hose 26b extending in a circumferential direction about a part of the interface plate 13. Accordingly, fluid drawn from the container into the fluid channel 26 flows from the filter outlet 25 along the radially extending hose 26a and into the hose 26b.

As illustrated, the hose 26b comprises a port 45a. In particular, the hose 26b comprises a plurality of ports 45a, 45b.

As illustrated in FIG. 15, the fluid channel 26 is defined between a first part 13a of the interface plate 13 and a second part 13b of the interface plate. Each port 45 comprises a septum seal 51 that sits in an opening in the first part 13a of the interface plate 13 so that it is pierceable from the exterior of the interface plate 13 to create a fluid path in communication with the fluid channel 26. Therefore, a waste consumable (5e, see FIG. 4) can be fluidly connected to the hose 26a via one of the ports 45a, 45b, for example, using a hollow needle as per the connector 19 illustrated in FIG. 5.

The plurality of ports 45a, 45b provides for a plurality of waste consumables 5e to be connected to the bioreactor 4 at different times without re-using a port 45a, 45b, in particular, the septum seal 51.

In some examples, the connector interfaces 21 may alternatively be used to deliver a fluid into the container 12 by connecting a delivery consumable to a port 45a, 45b. The fluid will flow through the fluid channel 26 and the filter 24 and into the container 12.

The interface plate 13 may include one or more separate ports 45c that are not connected to the fluid channel 26. The separate ports 45c can be used to input or extract fluid without the fluid passing through the filter 24.

As described above, each connector interface 21 may comprise a septum seal. In alternative examples each connector interface 21 may comprise a valve or cap or other openable closure.

As shown in FIG. 14, the plurality of ports 45a, 45b are located at different positions along the fluid channel 26, and are therefore located at different distances from the filter outlet 25 along the fluid channel 26. In particular, the port 45a is located closer to the filter outlet 25 along the fluid channel 26 than the port 45b. Therefore, when using port 45a fluid egressing from, for flowing toward, the container will not pass port 45b. This may be advantageous if port 45b has been used before as the port 45b may not reliably and totally seal after use, so sterility can be improved.

In addition, in some examples it may be advantageous to flush the fluid channel 26. For example, after extraction of waste fluid it may be advantageous to flush a clean fluid through the fluid channel 26. Accordingly, the clean fluid can be delivered to a port 45b that is a further distance from the filter outlet 25 than the port 45a used for waste fluid extraction.

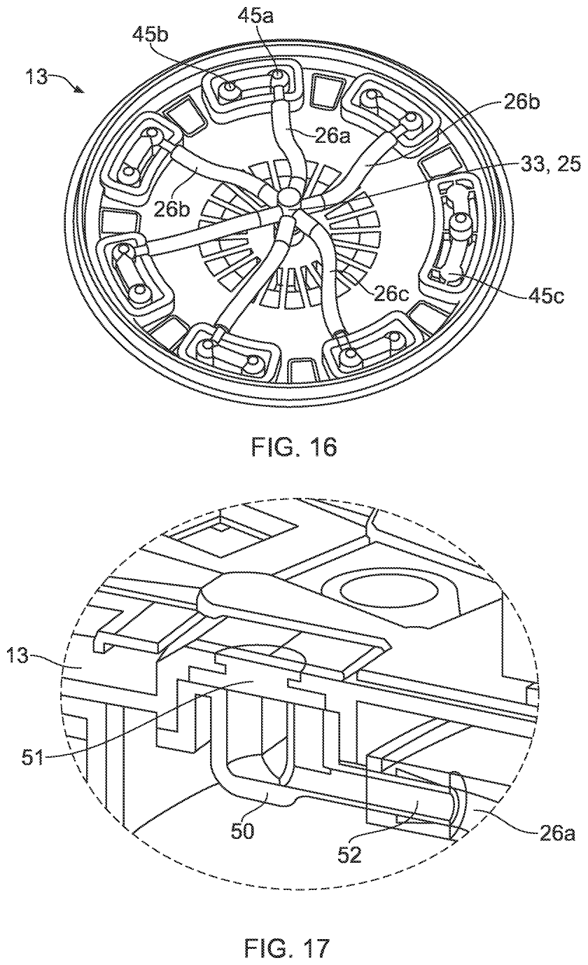

FIG. 16 illustrates a further example of the fluid channel 26 in the interface plate 13. In this example the fluid channel 26 is formed of one or more hoses 26a, 26b, 26c extending between the threaded spigot 33 instead at the filter outlet 25 and one or more ports 45. As shown in FIG. 16, the ports 45 are arranged circumferentially about an outer part of the bioreactor interface plate 13. As shown, each hose 26a, 26b, 26c is connected to more than one port 45, in this example a first port 45a and a second port 45b. As described previously, each port 45 may be associated with a connector interface (21, see FIG. 4) for connection to a consumable, such as a waste consumable (5e, see FIG. 4).

In the illustrated example the first port 45a is located between the second port 45b and the filter outlet 25 along the fluid path. Therefore, when using port 45a fluid egressing from, or flowing toward, the container will not pass port 45b. This may be advantageous if port 45b has been used before as the port 45b may not reliably and totally seal after use, so sterility can be improved.

In addition, in some examples it may be advantageous to flush the hose 26a. For example, after extraction of waste fluid it may be advantageous to flush a clean fluid through the hose 26a. Accordingly, the clean fluid can be delivered to a port 45b that is a further distance from the filter outlet 25 than the port 45a used for waste fluid extraction.

As illustrated in FIG. 17, each port 45 comprises a connector 50, for example, a spigot 52, for attachment of the hose 26a and a septum seal 51 that is pierceable from the exterior of the interface plate 13 to create a fluid path in communication with the hose 26a. The septum seal 51 is retained between the interface plate 13 and the connector 50. Therefore, a waste consumable (5e, see FIG. 4) can be fluidly connected to the hose 26a via one of the ports 45a, 45b, for example, using a hollow needle as per the connector 19 illustrated in FIG. 5.

The plurality of ports 45a, 45b provides for a plurality of waste consumables 5e to be connected to the bioreactor 4 at different times without re-using a port 45a, 45b, in particular, a septum seal.

In some examples, the connector interfaces 21 may alternatively be used to deliver a fluid into the container 12 by connecting a delivery consumable to a port 45a, 45b. The fluid will flow through the hose 26a, 26b, 26c and the filter 24 and into the container 12.

The interface plate 13 may include one or more separate ports 45c that are not connected to a hose 26c. The separate ports 45c can be used to input or extract fluid without the fluid passing through the filter 24.

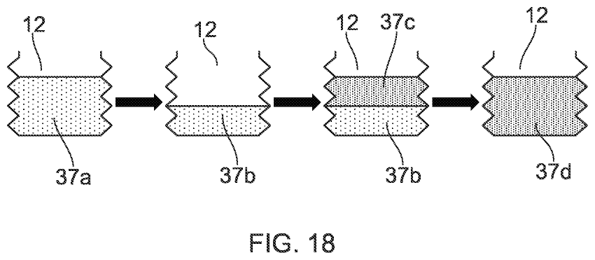

FIG. 18 illustrates a method of extracting a waste fluid from the container 12 of the bioreactor 4. As shown, initially the container 12 holds an initial cell suspension 37a that comprises cells and a fluid. Then, waste fluid is egressed from the container 12 as per the examples described above, in particular, by filtering cells from the fluid and extracting a waste fluid that does not comprise cells. The waste fluid is egressed through the filter 24 and into the waste consumable 5e as described above. The container 12 then holds a concentrated cell suspension 37b. Next, a flushing media 37c is delivered into the container 12. The flushing media 37c is delivered into the container 12 via the filter 24. The flushing media 37c acts to dislodge cells that may be caught on the filter 24, moving those cells back into the main fluid in the container 12. The mixed cell suspension 37d thereby comprises the cells and the flushing media. The flushing media 37c may comprise a lower volume than the waste fluid that is extracted, so that the mixed cell suspension 37d has a higher concentration than the initial cell suspension 37a.

Generally, it will be appreciated by persons skilled in the art that the above embodiments have been described by way of an example only and not in any limitative sense, and that various alternations and modifications are possible without departing from the scope of the disclosure as defined by the appended claims. Various modifications to the detailed designs as described above are possible, for example, variations may exist in shape, size, arrangement, assembly, sequence or the like. For example, any one of the enclosures, planar interfaces, component retaining elements or the like may be used in any suitable combination. Moreover, whilst the present disclosure has been described in relation to an automated process, it will be appreciated by persons skilled in the art that a user may manually, or semi-automatedly, undertake one or more of the above process steps.

1. A bioreactor interface plate assembly for attachment to a bioreactor container holding a cell suspension, the bioreactor interface plate assembly comprising:
a bioreactor inter plate including a port formed therein for egress of fluid from the bioreactor container; and
a filter attached to the bioreactor interface plate in fluid communication with the port, the filter positioned to be located within an internal volume of the bioreactor container while the bioreactor container is connected to the bioreactor interface plate, wherein the filter is configured to retain cells from the cell suspension in the internal volume when the fluid egresses from the bioreactor container via the port.

2. The bioreactor interface plate assembly of claim 1, wherein the filter comprises a filter housing attached to the bioreactor interface plate, and a filter member attached to the filter housing.

3. The bioreactor interface plate assembly of claim 2, wherein the filter housing is fixedly mounted to the bioreactor interface plate.

4. The bioreactor interface plate assembly of claim 3, wherein the filter is housing is fixedly mounted to a central portion of the bioreactor interface plate, the filter positioned to be disposed centrally within the bioreactor container while the bioreactor container is connected to the bioreactor interface plate.

5. The bioreactor interface plate assembly of claim 2, wherein the filter housing is suspended from the bioreactor interface plate by a flexible hose.

6. The bioreactor interface plate assembly of claim 2, wherein an internal surface of the filter housing and the filter member define a cavity therebetween, and wherein the port is in fluid communication with the cavity.

7. The bioreactor interface plate assembly of claim 6, wherein the internal surface of the filter housing comprises a taper.

8. The bioreactor interface plate assembly of claim 7, wherein the filter further comprises spacer ribs disposed between the internal surface of the filter housing and the filter member.

9. The bioreactor interface plate assembly of claim 2, wherein the filter housing further comprises an external surface facing the bioreactor interface plate, and wherein the external surface includes a frustrum-shape with a convex surface facing the bioreactor interface plate.

10. The bioreactor interface plate assembly of claim 1, wherein the port is adapted to connect to a fluid waste container to receive the fluid egressed from the bioreactor container.

11. The bioreactor interface plate assembly of claim 10, wherein the port comprises a septum seal.

12. The bioreactor interface plate assembly of claim 10, further comprising a fluid channel extending through the bioreactor interface plate between the filter and the port.

13. The bioreactor interface plate assembly of claim 12, wherein the bioreactor interface plate comprises a plurality of ports arranged along the fluid channel.

14. The bioreactor interface plate assembly of claim 2, wherein the filter member is chosen from among a filter membrane and a depth filter.

15. The bioreactor interface plate assembly of claim 1, further comprising an attachment member connected to the bioreactor interface plate and configured for attachment of the bioreactor interface plate to the bioreactor container and to seal the bioreactor container with the bioreactor interface plate.

16. A bioreactor comprising:
a bioreactor container defining an internal volume for holding a fluid cell suspension; and
a bioreactor interface plate including a port formed therein for egress of fluid from the bioreactor container; and
a filter attached to the bioreactor interface plate in fluid communication with the port, the filter positioned to be located within an internal volume of the bioreactor container while the bioreactor container is connected to the bioreactor interface plate, wherein the filter is configured to retain cells from the cell suspension in the internal volume when the fluid egresses from the bioreactor container via the port.

17. The bioreactor of claim 16, wherein the bioreactor container comprises a base section and a side wall, the bioreactor interface plate being attached to the side wall opposite to the base section.

18. The bioreactor of claim 17, wherein the side wall is at least one of compressible and extendible.

19. A method of extracting a fluid from a cell suspension held in a bioreactor container defining an internal volume for holding a fluid cell suspension, the method comprising:
positioning a bioreactor interface plate over the bioreactor container and at least partially submerging a filter in the cell suspension held in the bioreactor container, the bioreactor interface plate including a port formed therein for egress of fluid from the bioreactor container and the filter attached to the bioreactor interface plate in fluid communication with the port, the filter positioned to be located within an internal volume of the bioreactor container while the bioreactor container is connected to the bioreactor interface plate, and
generating a pressure differential to move fluid through the filter and the port to a separate container.

20. The method of claim 19, wherein at least partially submerging the filter in the cell suspension held in the bioreactor container comprises moving or deploying the filter.

21. The method of claim 19, wherein at least partially submerging the filter in the cell suspension held in the bioreactor container comprises compressing the bioreactor container to raise a fluid level of the cell suspension within the bioreactor container.

22. The method of claim 19, wherein generating a pressure differential to move the fluid through the filter and the outlet to a container comprises applying suction to the port to draw the fluid from the bioreactor container.

23. The method of claim 22, wherein applying suction to the port comprises expanding the container.

24. The method of claim 19, further comprising delivering a flushing fluid to the bioreactor container after extracting the fluid, the flushing fluid being delivered to the bioreactor container through the filter.
